(19) United States
(12) Patent Application Publication
(10) Pub. No.: US 2026/0198467 A1
(43) Pub. Date: Jul. 16, 2026

(54) METHOD FOR CONSTRUCTING MOUSE MODEL FOR RAPID DEGRADATION OF MSTN PROTEIN

(71) Applicant: Northwest A&F University, Xianyang City (CN)

(72) Inventors: Gang REN, Xianyang City (CN); Xiaobei Zhu, Xianyang City (CN); Yuwen Wei, Xianyang City (CN); Linqian Pan, Xianyang City (CN); Zixiao Liu, Xianyang City (CN); Shengxuan Wang, Xianyang City (CN); Shuhan Li, Xianyang City (CN); Yijie Fan, Xianyang City (CN)

(21) Appl. No.: 19/276,352

(22) Filed: Jul. 22, 2025

(30) Foreign Application Priority Data
Jan. 14, 2025 (CN) 202510056799.4

(51) Int. Cl. A01K 67/0278; C12N 9/22; C12N 15/11

(52) U.S. Cl. CPC A01K 67/0278; C12N 9/226; C12N 15/111; A01K 2227/105; A01K 2267/0393; C12N 2310/20

(57) ABSTRACT

A method for constructing a mouse model for rapid degradation of Myostatin (MSTN) protein is provided. The method includes: inserting sequentially a 5′ homologous arm, 3 X (Glu-Ala-Ala-Ala-Lys) (3xEAAAK), an FK506-binding protein (FKBP1A) gene synonymous mutation sequence, 3xEAAAK, a red fluorescent protein monomeric Cherry (mCherry) sequence and a 3′ homologous arm into a Donor vector backbone to obtain a Donor recombinant vector; designing a Guide Ribonucleic Acid (gRNA) based on a third exon of the Mstn gene; mixing the gRNA, CRISPR-associated protein 9 (Cas9) protein, and the Donor recombinant vector and co-injecting a mixture of the above into mouse fertilized eggs, culturing the fertilized eggs in vitro and transplanting to obtain the F0 generation mice; and hybridizing the F0 generation mice with wild-type mice, or further breeding hybridized offspring to obtain a knock-in mouse model.

### CROSS-REFERENCE TO RELATED APPLICATIONS

This application claims priority to Chinese Patent Application No. 202510056799.4, filed on Jan. 14, 2025, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a) ), submitted via the USPTO patent electronic filing system or on one or more read-only optical discs (see Rule 1.52(e)(8) ), identifying the names of each file, the date of creation of each file, and the size of each file in bytes as follows:
File name: SequenceListing.xml
Creation date: Jul. 18, 2025
Byte size: 30,574

### TECHNICAL FIELD

The present disclosure belongs to the field of genetic engineering, and relates to the establishment of a mouse Myostatin-FK506-binding protein-monomeric Cherry (MSTN-FKBP-mCherry) knock-in model based on Clustered Regularly Interspaced Short Palindromic Repeats/CRISPR-associated protein 9 (CRISPR/Cas9)-mediated gene editing technology, and in particular to a method for constructing transgenic mice with human FK506-Binding Protein 1A (FKBP1A) gene sequences knocked in.

### BACKGROUND

Over the past few decades, molecular regulation technologies have advanced continuously and become important means for studying gene functions in various biological contexts. Broadly, these technologies may be classified based on their targets: Deoxyribonucleic Acid (DNA), Ribonucleic Acid (RNA), or proteins. However, since proteins play key roles in many biological processes, interventions targeting only DNA and RNA have some limitations. These factors may affect experimental design and hinder accurate understanding of protein functions in scenarios requiring rapid and reversible intervention. Therefore, technologies that may directly target and regulate protein levels have become particularly attractive, as they may offer faster resolution of action time than DNA/RNA-targeting technologies. The latest degradation tag (dTAG) protein degradation system provides the possibility of targeting any intracellular protein in a rapid, inducible, and reversible manner.

The dTAG system is an innovative target confirmation method that manipulates intracellular protein degradation mechanisms using special bifunctional small molecules to achieve degradation of specific proteins. Developed by Dr. Behnam Nabet and his team at the Dana Farber Cancer Institute, this system not only has broad application prospects but also is one of the effective strategies to achieve the targeted protein degradation (TPD). The dTAG system induces the degradation of the target protein by utilizing the endogenous proteasome degradation pathway in cells through fusion of the target protein with an FK506-binding protein (FKBP) tag and subsequent application of the specific small molecule degrader dTAG. Existing technologies have proposed two methods for constructing targeted protein degradation: knock-in of genome-specific FKBP tag by Clustered Regularly Interspaced Short Palindromic Repeats/CRISPR-associated protein 9 (CRISPR-Cas9) system or fusion of FKBP tag by exogenous expression of lentivirus system to the target, and the target protein is expressed as a chimera with FKBP tag mutant. After adding the dTAG ligand, a ternary complex is formed between the target protein fused with the FKBP tag and the E3 ligase, triggering polyubiquitination of the target protein, leading to the degradation of the target protein by the proteasome. This enables rapid and selective processing of the tagged protein. To verify the potential of the dTAG system in regulating protein levels in live mice, existing technologies injected human leukemia cell line MV4-11 carrying a luciferase (luc-FKBP12F36V) reporter gene with FKBP tag into the bone marrow of mice. After treatment with dTAG molecules, it is observed that the activity of reporter gene in mice decreased significantly within 4 hours and rose again within 28 hours. This discovery confirmed that the dTAG system enables rapid and reversible assessment of target protein function in live mice.

Myostatin (MSTN) is a secreted signal transduction molecule, which is initially discovered during the screening of new members of the Transforming Growth Factor (TGF)-β superfamily. MSTN is primarily expressed in skeletal muscle and acts as a negative regulator of muscle growth and development. Multiple species (including cattle, sheep, dogs, rabbits, rats, pigs, goats, humans and mice) have been proven that MSTN deficiency may lead to muscle hypertrophy, increased muscle fiber numbers, and enlarged muscle fiber size without causing severe adverse consequences. Additionally, in the animal model of cancer malignant transformation, it has been proved that inhibiting MSTN signal transduction or Mstn gene mutation may counteract the loss of muscle mass and strength and prolong the survival time without affecting tumor growth. Therefore, extensive efforts have been made to develop effective strategies for blocking MSTN expression to increase muscle mass in animals. People have devoted a great deal of effort in developing drugs that may regulate MSTN signal transduction for clinical applications. However, MSTN inhibitors tested in clinical trials to date have been limited to protein drugs, such as neutralizing antibodies, peptibodies, monomers, or decoy receptors. In the mouse genome, the Mstn gene is located on chromosome 1, with a total length of 6.44 Kilobase (kb), containing 3 exons. The start codon Adenine-Thymine-Guanine (ATG) is located in the first exon, and the stop codon Thymine-Guanine-Adenine (TGA) is located in the third exon.

The degradation of target protein by the dTAG system is rapid, adjustable, and reversible, and it has been widely used in various cell lines and mouse models to study target protein functions. The successful construction of Myostatin-FK506-binding protein-monomeric Cherry (MSTN-FKBP-mCherry) knock-in mice is crucial for studying MSTN protein functions and discovering and verifying new targets by using the dTAG system. Compared to drug inhibitors, dTAG small molecule degraders offer significant advantages, including simpler production processes, more convenient clinical applications, and the ability to overcome resistance to inhibitors; the degree of protein knock-down may be adjusted by changing the dosage; and the effects may occur more rapidly.

In summary, the construction of MSTN-FKBP-mCherry fusion protein-expressing mice is particularly critical for achieving rapid and reversible degradation and clearance of MSTN protein in mice.

### SUMMARY

The objective of the disclosure is to provide a method for constructing a mouse model for rapid degradation of Myostatin (MSTN) protein. This method utilizes Clustered Regularly Interspaced Short Palindromic Repeats/CRISPR-associated protein 9 (CRISPR/Cas9) technology and microinjection technology to obtain a mouse model that may stably and efficiently express the Myostatin-FK506-binding protein-monomeric Cherry (MSTN-FKBP-mCherry) fusion protein.

To achieve the above objective, the disclosure adopts the following technical schemes.

The present disclosure provides a method for constructing a mouse model for rapid degradation of MSTN protein, including following steps:
inserting sequentially a 5′ homologous arm, 3x (Glu-Ala-Ala-Ala-Lys) (3xEAAAK), an FK506-Binding Protein 1A (FKBP1A) gene synonymous mutation sequence, 3xEAAAK, a red fluorescent protein mCherry sequence and a 3′ homologous arm into a Donor vector backbone to obtain a Donor recombinant vector; where a nucleotide sequence of the FKBP1A gene synonymous mutation sequence is as shown in Sequence Identification Number (SEQ ID NO.) 4, a nucleotide sequence of 3xEAAAK is as shown in SEQ ID NO. 3, a nucleotide sequence of the red fluorescent protein mCherry is as shown in SEQ ID NO. 5, a nucleotide sequence of the 5′ homologous arm is as shown in SEQ ID NO. 2, and a nucleotide sequence of the 3′ homologous arm is as shown in SEQ ID NO. 6;
designing a Guide Ribonucleic Acid (gRNA) based on a third exon of the Mstn gene on mouse chromosome 1, where a nucleotide sequence of the gRNA is as shown in SEQ ID NO. 1;
mixing the gRNA, CRISPR-associated protein 9 (Cas9) protein, and the Donor recombinant vector and co-injecting a mixture of the above into mouse fertilized eggs to knock the FKBP1A gene synonymous mutation sequence and the red fluorescent protein mCherry sequence into a mouse genome;
culturing the fertilized eggs after injection in vitro and transplanting into surrogate mice to continue development until birth of young mice, thus obtaining F0 generation mice; and
hybridizing the F0 generation mice with wild-type mice, or further breeding hybridized offspring to obtain a mouse expressing MSTN-FKBP-mCherry fusion protein, namely, a mouse model for rapid degradation of the MSTN protein.

The Thymine-Guanine-Adenine (TGA) stop codon of mouse Mstn gene is located in exon 3. After the knock-in sequence is introduced, the TGA stop codon will be replaced by “3xEAAAK-mutant human FKBP1A CDS (excluding Adenine-Thymine-Guanine (ATG) start codon) −3xEAAAK-mCherry”.

As an optional embodiment of the present disclosure, the Donor recombinant vector is constructed by following steps:
using a Bacterial Artificial Chromosome (BAC) plasmid as a template, amplifying fragments including a 5′ homologous arm and a 3′ homologous arm from the mouse genome respectively to obtain a fragment-1 and a fragment-3;
connecting 3xEAAAK, the FKBP1A gene synonymous mutation sequence, 3xEAAAK, and the red fluorescent protein mCherry sequence sequentially to obtain a fragment-2; and
ligating the fragment-1, the fragment-2, the fragment-3, and the Donor vector backbone, incubating a resulting ligation product, transforming into competent cells, culturing, and extracting to obtain the Donor recombinant vector, where a nucleotide sequence of the Donor recombinant vector is as shown in SEQ ID NO. 21.

In an embodiment, primer sequences for amplifying the fragment including the 5′ homologous arm are as shown in SEQ ID NO. 7 and SEQ ID NO. 8, and primer sequences for amplifying the fragment including the 3′ homologous arm are as shown in SEQ ID NO. 9 and SEQ ID NO. 10.

In an embodiment, when connecting the fragment-1, the fragment-2, the fragment-3, and the Donor vector backbone, a 20 microliter (μL) ligation system includes: 80.0 nanogram (ng) of the Donor vector backbone, 7.19 ng of the fragment-1, 21.28 ng of the fragment-2, 27.54 ng of the fragment-3, and 10 μL of NEBuilder® HiFi DNA Assembly MasterMix.

In an embodiment, the amplification system is as follows: 1 uL of template DNA, 10 uM Forward and Reverse primers 1 uL, 12.5 uL of 2xPhanta Max Master Mix (Dye Plus), and supplemented by water to 25 uL.

In an embodiment, the amplification program is as follows: pre-denaturation at 95 degrees Celsius (° C.) for 3 minutes (min); 35 cycles of denaturation at 95° C. for 15 seconds (s), annealing at 58° C. for 35 s, and extension at 72° C. for 30-60 s; and final extension at 72° C. for 5 min.

As an optional embodiment of the present disclosure, the competent cells are DH5-alpha competent cells.

As an optional embodiment of the present disclosure, the gRNA and the Cas9 protein are mixed and incubated in a metal bath, then the Donor recombinant vector is added, mixed evenly, and co-injected into the mouse fertilized eggs.

In an embodiment, the incubation is performed in a 25° C. metal bath for 10 min.

As an optional embodiment of the present disclosure, a concentration of the gRNA is 90-110 picomole per microliter (pmol/μL), a concentration of the Cas9 protein is 18-22 micromolar (μM), a concentration of the Donor recombinant vector is 12-18 nanogram per microliter (ng/μL), a volume ratio of the gRNA to the Cas9 protein is 3-5:1, and a total volume of a mixture of the gRNA and Cas9 protein with the Donor recombinant vector is 20 μL.

The second aspect of the disclosure provides a mouse model for rapid degradation of the MSTN protein constructed by the above method.

As an optional embodiment of the present disclosure, obtained MSTN-FKBP-mCherry knock-in mice are administered with degradation tag (dTAG) to achieve rapid and reversible degradation and clearance of the MSTN protein in the mice.

The beneficial effects of the disclosure are as follows.

The disclosure is based on CRISPR/Cas9-mediated gene editing technology to knock in the human FKBP1A protein coding sequence and the red fluorescent protein mCherry sequence (labeled as the FKBP-mCherry tag sequence) into the mouse Mstn gene, and synonymous mutations of FKBP1A are introduced to avoid interference from endogenous splicing sites and off-target effects and the like, thereby establishing a mouse model for rapid degradation of MSTN protein, i.e., a mouse MSTN-FKBP-mCherry knock-in model.

The fusion expression of mCherry fluorescent protein after FKBP tag plays an indication role, and the degradation of target protein may be determined by observing whether the fluorescence weakens. This mouse MSTN-FKBP-mCherry knock-in model may stably and efficiently express the MSTN protein fused with the FKBP-mCherry tag (abbreviated as the MSTN-FKBP-mCherry fusion protein). Additionally, the modeling process of the mouse MSTN-FKBP-mCherry knock-in model in the disclosure has the advantage of a short operation cycle.

The present disclosure promotes the expression of the MSTN-FKBP-mCherry fusion protein by adding the fusion protein linker 3xEAAAK sequence between the Mstn gene, the FKBP1A synonymous mutation sequence, and the mCherry insertion sequence.

The present disclosure achieves rapid and reversible degradation and clearance of the MSTN protein in mice by administering degradation tag (dTAG) to the obtained knock-in mice.

The present disclosure relates to F0-generation positive mice (i.e., successfully knocked-in positive F0 heterozygous mice) constructed using CRISPR/Cas9 technology. Due to variable cutting efficiency and potential non-homologous repair, different genetic lineages may occur, the F0-generation positive mice are bred with wild-type mice to obtain F1-generation positive mice with stable genotypes.

### BRIEF DESCRIPTION OF THE DRAWINGS

FIG. 1 is a schematic diagram of transgenic mouse models expressing the FK506-binding protein-monomeric Cherry (FKBP-mCherry) tag constructed by using Clustered Regularly Interspaced Short Palindromic Repeats/CRISPR-associated protein 9 (CRISPR/Cas9) technology in an embodiment of the present disclosure.

FIG. 2 is a plasmid map of the Donor vector used for homologous recombination repair.

FIG. 3 is a breeding scheme for F0 generation mice in an embodiment of the present disclosure.

FIG. 4 is a genotyping Polymerase Chain Reaction (PCR) identification strategy diagram for F0 and F1 generation mice in an embodiment of the present disclosure.

FIG. 5A is an electrophoresis diagram of genotyping PCR identification for F1 generation mice in the positive genome of 5′ homologous arm (HA) homologous recombination in an embodiment of the present disclosure.

FIG. 5B is an electrophoresis diagram of genotyping PCR identification for F1 generation mice in the positive genome of 3′ homologous arm (HA) homologous recombination in an embodiment of the present disclosure.

FIG. 6 is a (Knock-in KI) sequence sequencing diagram of F1 generation mice in an embodiment of the present disclosure.

FIG. 7 is an electrophoresis diagram of genotyping PCR identification for F2 generation mice in an embodiment of the present disclosure.

FIG. 8 is a Western blot identification diagram for detecting expressed Myostatin (MSTN) protein in an embodiment of the present disclosure.

### DETAILED DESCRIPTION OF THE EMBODIMENTS

The present disclosure will be further described in detail with the attached drawings and embodiments. The embodiments are only used to explain the disclosure, and are not intended to limit the scope of protection of the disclosure.

### Embodiment 1

Establishment of Myostatin-FK506-binding protein-monomeric Cherry (MSTN-FKBP-mCherry) knock-in model of C57BL/6J mice by Clustered Regularly Interspaced Short Palindromic Repeats/CRISPR-associated protein 9 (CRISPR/Cas9)-mediated gene editing technology

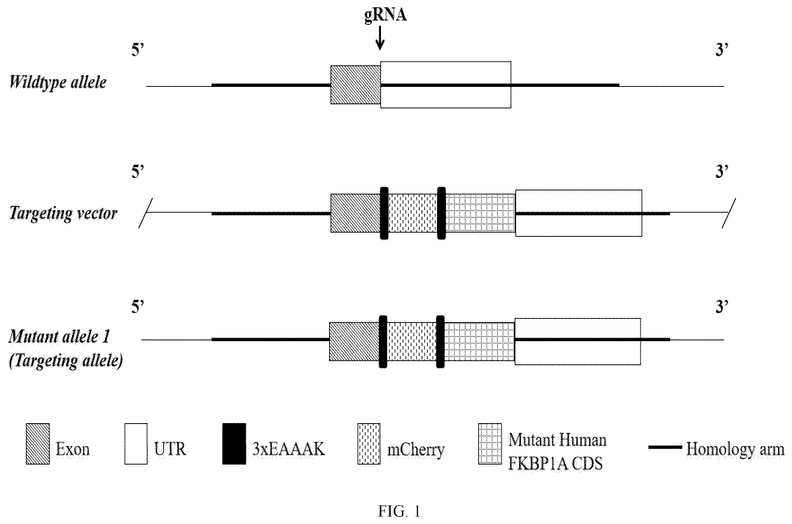

For the human gene FK506-Binding Protein 1A (FKBP1A), a mouse transgenic strategy is designed. The CDS sequence of FKBP1A is knocked into the third exon of the Mstn gene (NCBI reference sequence: NM_010834.3) on mouse chromosome 1 using CRISPR/Cas9 technology, as shown in FIG. 1. The specific steps are as follows:
1. Guide Ribonucleic Acid (gRNA) is designed based on the CRISPR/Cas9 system. The sequence of the third exon specific gRNA action site of Mstn gene is shown in Table 1.

TABLE 1
gRNA action site

| Sequence (5′-3′) | Sequence Number |
|---|---|
| GGGAAGACCTTCCATGACTTGGG | Sequence Identification |
| | Number |
| | (SEQ ID NO. ) 1 |

Note:
the italicized bases in the sequence are the protospacer
adjacent motif (PAM).

2. The Donor vector is designed based on the principle of homologous recombination repair and combined with the knocked-in target sequence (referred to as the knock-in sequence). The Donor vector includes the 5′ homologous arm, 3x (Glu-Ala-Ala-Ala-Lys) (3xEAAAK), the synonymous mutation sequence of the FKBP1A gene (labeled as Mutant Human FKBP1A CDS-p. F37V), 3REAAAK, the red fluorescent protein mCherry sequence, and the 3?homologous arm. The main components are shown in Table 2, and the nucleotide sequences of the corresponding elements are shown in Table 3.

TABLE 2
Main components of the Donor vector

| | Fragment |
|---|---|
| Component Name | Length (bp) |
| 3xEAAAK | 45 |
| Mutant Human FKBP1A CDS (p.F37V (TTT to GTT)) | 321 |
| mCherry | 708 |

TABLE 3
Partial nucleotide sequences of the Donor vector

| | Sequence | |
|---|---|---|
| Component Name | Number | Sequence (5′-3′) |
| 5′ | SEQ ID | AATCCCTTTTTAGAAGTCAAGGTGACAGACACACCCA |
| homologous | NO. 2 | AGAGGTCCCGGAGAGACTTTGGGCTTGACTGCGATGA |
| arm | | GCACTCCACGGAATCCCGGTGCTGCCGCTACCCCCTC |
| | | ACGGTCGATTTTGAAGCCTTTGGATGGGACTGGATTA |
| | | TCGCACCCAAAAGATATAAGGCCAATTACTGCTCAGG |
| | | AGAGTGTGAATTTGTGTTTTTACAAAAATATCCGCATA |
| | | CTCATCTTGTGCACCAAGCAAACCCCAGAGGCTCAGC |
| | | AGGCCCTTGCTGCACTCCGACAAAAATGTCTCCCATT |
| | | AATATGCTATATTTTAATGGCAAAGAACAAATAATAT |
| | | ATGGGAAAATTCCAGCCATGGTAGTAGACCGCTGTGG |
| | | GTGCTCA |
| 3xEAAAK | SEQ ID | GAAGCTGCGGCAAAAGAAGCAGCGGCTAAAGAAGCG |
| | NO. 3 | GCGGCAAAA |
| Mutant | SEQ ID | GGAGTGCAGGTGGAAACCATCTCCCCAGGAGACGGG |
| Human | NO. 4 | CGCACCTTCCCCAAGCGCGGCCAGACCTGCGTGGTGC |
| FKBP1A | | ACTACACCGGGATGCTTGAAGATGGAAAGAAAGTTG |
| CDS (p.F37V | | ATTCCTCCCGGGACAGAAACAAGCCCTTTAAGTTTAT |
| (TTT to | | GCTAGGCAAGCAGGAGGTGATCCGAGGCTGGGAAGA |
| GTT)) | | AGGGGTTGCCCAGATGAGTGTGGGTCAGAGAGCCAA |
| | | ACTGACTATATCTCCAGATTATGCCTATGGTGCCACTG |
| | | GGCACCCAGGCATCATCCCACCACATGCCACTCTCGT |
| | | CTTCGATGTGGAGCTTCTAAAACTGGA A |
| mCherry | SEQ ID | GTGAGCAAGGGCGAGGAGGATAACATGGCCATCATC |
| | NO. 5 | AAGGAGTTCATGCGCTTCAAGGTGCACATGGAGGGCT |
| | | CCGTGAACGGCCACGAGTTCGAGATCGAGGGCGAGG |
| | | GCGAGGGCCGCCCCTACGAGGGCACCCAGACCGCCA |
| | | AGCTGAAGGTGACCAAGGGTGGCCCCCTGCCCTTCGC |
| | | CTGGGACATCCTGTCCCCTCAGTTCATGTACGGCTCCA |
| | | AGGCCTACGTGAAGCACCCCGCCGACATCCCCGACTA |
| | | CTTGAAGCTGTCCTTCCCCGAGGGCTTCAAGTGGGAG |
| | | CGCGTGATGAACTTCGAGGACGGCGGCGTGGTGACCG |
| | | TGACCCAGGACTCCTCCCTGCAGGACGGCGAGTTCAT |
| | | CTACAAGGTGAAGCTGCGCGGCACCAACTTCCCCTCC |
| | | GACGGCCCCGTAATGCAGAAGAAAACCATGGGCTGG |
| | | GAGGCCTCCTCCGAGCGGATGTACCCCGAGGACGGCG |
| | | CCCTGAAGGGCGAGATCAAGCAGAGGCTGAAGCTGA |
| | | AGGACGGCGGCCACTACGACGCTGAGGTCAAGACCA |
| | | CCTACAAGGCCAAGAAGCCCGTGCAGCTGCCCGGCGC |
| | | CTACAACGTCAACATCAAGTTGGACATCACCTCCCAC |
| | | AACGAGGACTACACCATCGTGGAACAGTACGAACGC |
| | | GCCGAGGGCCGCCACTCCACCGGCGGCATGGACGAGC |
| | | TGTACAAGTAA |
| 3′ | SEQ ID | GCTTTGCATTAGGTTAGAAATTTCGCAAGTCATGGAA |
| homologous | NO. 6 | GGTCTTCCCCTCAATTTCGAAACTGTGAATTCAAGCAC |
| arm | | CACAGGCTGTAGGCCTTGAGTATGCTCTAGTAACGTA |
| | | AGCACAAGCTACAGTGTATGAACTAAAAGAGAGAAT |
| | | AGATGCAATGGTTGGCATTCAACCACCAAAATAAACC |
| | | ATACTATAGGATGTTGTATGATTTCCAGAGTTTTTGAA |
| | | ATAGATGGAGATCAAATTACATTTATGTCCATATATGT |
| | | ATATTACAACTACAATCTAGGCAAGGAAGTGAGAGCA |
| | | CATCTTGTGGTCTGCTGAGTTAGGAGGGTATGATTAA |
| | | AAGGTAAAGTCTTATTTCCTAACAGTTTCACTTAATAT |
| | | TTACGGAAGAATCTATATGTAGCCTTTGTAAAGTGTA |
| | | GGATTGTTATCATTTAAAAACATCATGTACACTTATAT |
| | | TTGTATTGTATACTTGGTAAGATAAAATTCCACAAAGT |
| | | AGGAATGGGGCCTTACATACACATTGCCATTCCTATT |
| | | ATAATTGGACAATCCACCACGGTGCTAATGCAGTGCT |
| | | GAATGGCTCCTACTGGACCTCTCGATAGAACACTCTA |
| | | CAAAGTACGAGTCTCTCTCTCCCTTCCAGGTGCATCTC |
| | | CACACACACAGCACTAAGTGTTCAATGCATTTTCTTTA |
| | | AGGAAAGAAGAATCTTTTTTTCTAGAGGTCAACTTTC |
| | | AGTCAACTCTAGCACAGCGGGAGTGACTGCTGCATCT |
| | | TAAAAGGCAGCCAAACAGTATTCATTTTTTAATCTAA |
| | | ATTTCAAAATCACTGTCTGCCTTTATCACATGGCAATT |
| | | TTGTGGTAAAATAATGGAAATGACTGGTTCTATCAAT |
| | | ATTGTATAAAAGACTCTGAAACAATTACATTTATATA |
| | | ATATGTATACAATATTGTTTTGTAAATAAGTGTCTCCT |
| | | TTTATATTTACTTTGGTATATTTTTACACTAATGAAATT |
| | | TCAAATCATTAAAGTACAAAGACATGTCATGTATCAC |
| | | AAAAAAGGTGACTGCTTCTATTTCAGAGTGAATTAGC |
| | | AGATTCAATAGTGGTCTTAAAACTCTGTATGTTAAGAT |
| | | TAGAAGGTTATATTACAATCAATTTATGTATTTTTTAC |
| | | ATTATCAACATTCACTTATGGTTTCATGGTGGCTGTAT |
| | | CTATGAATGTGGCTCCCAGTCAAATTTCAATGCCCCAC |
| | | CATTTTAAAAATTACAAGCATTACTAAACATACCAAC |
| | | ATGTATCTAAAGAAATACAAATATGGTATCTCAATAA |
| | | CAGCTACTTTTTTATTTTATAATTTGACAATGAATACA |
| | | TTTCTTTTATTTACTTCAGTTTTATAAATTGGAACTTTG |
| | | TTTATCAAATGTATTGTACTCATAGCTAAATGAAATTA |
| | | TTTCTTACATAAAAATGTGTAGAAACTATAAATTAAA |
| | | GTGTTTTCACATTTTTGAAAGGC |

Note:
the underlined bases are the introduced synonymous mutations. The original
sequence is TTT, and the mutated sequence is GTT.

3. By using In-Fusion technology, the above 3REAAAK, Mutant Human FKBP1A CDS-p.F37V (TTT to GTT) and mCherry are assembled into the Donor vector. Specifically, sequentially inserting the 5′ homologous arm, the fusion protein linker 3REAAAK, the FKBP1A gene synonymous mutation sequence, 3REAAAK, the red fluorescent protein mCherry sequence, and the 3?homologous arm into the backbone of the Donor vector. The specific steps are as follows:
(1) using high-fidelity Thermus aquaticus (Taq) Deoxyribonucleic Acid (DNA) polymerase and the Bacterial Artificial Chromosome (BAC) (RPCI-23.C) plasmid (thermoFisher scientific) as a template, fragment-1 (containing the 5′ homologous arm) and fragment-3 (containing the 3′ homologous arm) containing homologous sequences from the mouse genome are amplified respectively. The amplification primer sequences are shown in Table 4, the amplification system is shown in Table 5, and the amplification program is shown in Table 6.

Synthesis of the 3xEAAAK-Mutant Human FKBP1A CDS (excluding the ATG start codon)-3xEAAAK-mCherry sequence (denoted as fragment-2) (Shanghai Sangon Biotech).

TABLE 4
Nucleotide sequences of amplification primers

| Primer | | Primer | |
|---|---|---|---|
| Name | Sequence (5′-3′) | Type | Sequence Number |
| 5′arm-F | TTCCTTTTCACACAGAATCCCTTTTTAGAAG | Forward | SEQ ID NO. 7 |
| | | primer | |
| 5′arm-R | TTCTTTTGCCGCAGCTTCTGAGCACCCACA | Reverse | SEQ ID NO. 8 |
| | | primer | |
| 3′arm-F | GAGCTGTACAAGTAAGCTTTGCATTAGGTTA | Forward | SEQ ID NO. 9 |
| | | primer | |
| 3′arm-R | ATTGAACACTTAGTGCTGTGTGTGTGGAGATGCACCT | Reverse | SEQ ID NO. 10 |
| | | primer | |

TABLE 5
Polymerase Chain Reaction (PCR) system

| Component | Amount (microliter (μL)) |
|---|---|
| Template DNA | 1 |
| Forward primer F (10 micromolar (μM)) | 1 |
| Reverse primer R (10 μM) | 1 |
| 2 × Phanta Max Master Mix (Dye Plus) | 12.5 |
| ddH₂O | 9.5 |
| Total | 25 |

TABLE 6
PCR program

| | Temperature (degrees | | |
|---|---|---|---|
| Step | Celsius (° C.)) | Time | Cycles |
| Pre-denaturation | 95 | 3 minutes (min) | 1 |
| Denaturation | 95 | 15 s | 35 |
| Annealing | 58 | 35 s | |
| Extension | 72 | 30-60 seconds per | |
| | | kilobyte (sec/kb) | |
| Final extension | 72 | 5 min | 1 |
| Storage | 4 | ∞ | 1 |

(2) The existing plasmid (customized from Cyagen, internal catalog number Cat: VB139; specifically modified from the commercial plasmid Addgene #50005 to provide the vector backbone) are digested with Acc65I (NEB, R0599) and Asc I (NEB, R0558) restriction enzymes. The digestion system is shown in Table 7. After gel electrophoresis of the digestion products, the target band is cut out, recovered and purified using a DNA gel recovery kit (OMEGA, D2000) to obtain the linearized plasmid (4201 base pair (bp), i.e., the vector backbone).

TABLE 7
Digestion system

| Component | Amount | |
|---|---|---|
| Plasmid DNA | 1.0 microgram (μg) | |
| 10 × rCutSmart ™ Buffer | 5.0 | μL |
| Asc I | 1 | μL |
| Acc65I | 0.5 | μL |

| | |
|---|---|
| ddH₂O | Up to 50.0 μL |

(3) Using the seamless cloning kit (NEB, E2621), the PCR-amplified fragments are connected with the linearized plasmid according to the reaction system shown in Table 8. The reaction is performed in a PCR instrument and incubated at 50° C. for 60 min; then it is placed on ice for transformation.

TABLE 8
In-Fusion system

| Component | Amount | |
|---|---|---|
| Linearized plasmid | 80.0 nanograms (ng) | |
| Fragment-1 | 7.19 | ng |
| Fragment-2 | 21.28 | ng |
| Fragment-3 | 27.54 | ng |
| NEBuilder HiFi DNA | 10 | μL |
| Assembly Master Mix | | |

| | |
|---|---|
| ddH₂O | Up to 20.0 μL |

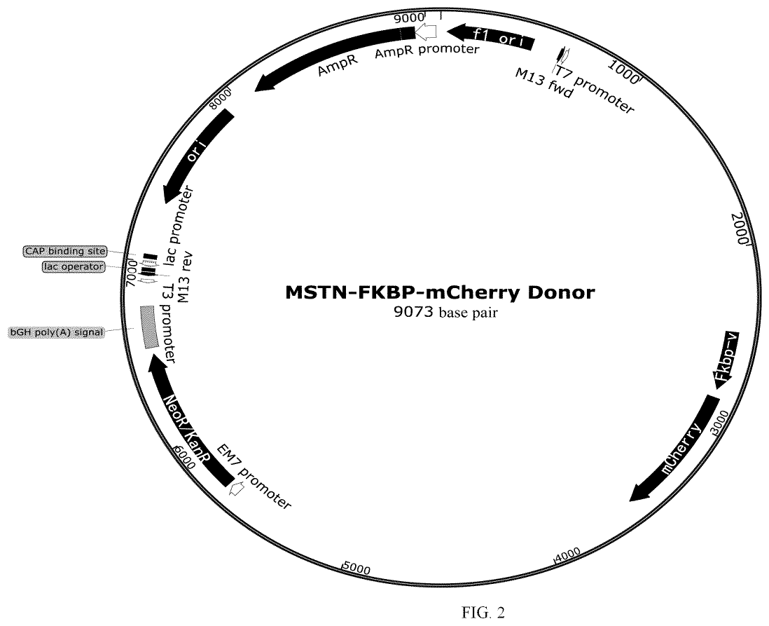

(4) The In-Fusion ligation products are transformed into DH5-alpha competent cells, then the competent cells are added into Luria-Bertani (LB) liquid medium and cultured at 37° C. in a shaker for 30 min; the obtained culture is coated on LB solid medium containing Ampicillin and cultured overnight; after ampicillin resistance (AmpR) screening, single colonies growing on the LB solid medium is selected into LB liquid medium for culture, then PCR identification of bacterial liquid is performed; after successful ligation is confirmed, the bacterial liquid is sent for sequencing; based on the sequencing results, single colonies with successful ligation and correct sequences are selected for shake the bacteria, then the bacterial liquid is collected for plasmid extraction to obtain the Donor recombinant vector. The plasmid map is shown in FIG. 2.

The nucleotide sequence of the Donor recombinant vector is as shown in SEQ ID NO. 21:

CTAAATTGTAAGCGTTAATATTTTGTTAAAATTCGCGTTAAATTTTTGTTA
AATCAGCTCATTTTTTAACCAATAGGCCGAAATCGGCAAAATCCCTTATAAATCAAA
AGAATAGACCGAGATAGGGTTGAGTGTTGTTCCAGTTTGGAACAAGAGTCCACTATT
AAAGAACGTGGACTCCAACGTCAAAGGGCGAAAAACCGTCTATCAGGGCGATGGCC
CACTACGTGAACCATCACCCTAATCAAGTTTTTTGGGGTCGAGGTGCCGTAAAGCAC
TAAATCGGAACCCTAAAGGGAGCCCCCGATTTAGAGCTTGACGGGGAAAGCCGGCG
AACGTGGCGAGAAAGGAAGGGAAGAAAGCGAAAGGAGCGGGCGCTAGGGCGCTGG
CAAGTGTAGCGGTCACGCTGCGCGTAACCACCACACCCGCCGCGCTTAATGCGCCG
CTACAGGGCGCGTCCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCC
AGTGAATTGTAATACGACTCACTATAGGGCGAATTGGGTACGGCGCGCCAAGCTAA
CCTTTGCCTCCTAGCTTATCTTTGGACTCCTTTAGCATACTTGCATAGGAATTAACTT
AGGCATTACCAATTAACTAAATAGTGAGTCAGTGATAGGACAAGACCTCATATATT
ATAAAAATAATAAAAAAATATTTAGCCCTCCAAAACCTGAGTTACTGTTCTCTGTTA
CTTTTATTTATTTATTTATCGGCTTTCAAAAACTTAACGTGAACATATGGTTATTTCC
ATCCACTGTCATAGTTTACACCAAAATATTGTACTTAGAAATAATAAGCCTGGTTAC
CTTTTAACCTTTTGATTGAACTTGGGAATATACAGTCTGGGAATCAGATCTGTTGTG
AGTGAAACCTGTCATTTGAAGTACTACGATTTCCTAAATGATCTATTTTCTTTATATA
TGCTACTTAAACACTACACTCTACAAATTATGTTCTCTTTGACAATATAGTCTCATGG
TAAAAGATAAAATTGCTATCAACTATTTAAGAACATTCTTTCTCATAGACAGTATTT
TAATTACCAAGGTTGCTAACAGGTTTTAATTATTTACAAGGTGGTTAGTGTGATATT
CCTTAGAGTTTTCTCTCTAAAAGGAAATGGTCCTAAATGCACTGGGAGATGGAAGCG
GTCATGCTGCATGCAAATTCTTCTCTTCCTTCTGTTCCTTCTGCTGTGGTCCTGAGGA
TTGACCCAGAACCTTGCACATGACGGGCAAACCCTGTTCCACCAAGCTCCATTCACT
CCCAGTTCATCACTTCTTATAAACCTCTTCTTTGCTCCTTTAAGGATTCTCTTTCCACG
CACTATACATACAGCCACTGTCATCATGCTAATTTCTCTCTTCCCACTGTAATCCATA
TAGTGCTCAGAAAGAGCTACTTTGCTCTGACTTTAATATGCACATTTCTTTCAGAAA
AGACCTAGGGGAGACCAGATGCGTACCTATCCATCCAAGAGCCATTAGTCACCTGC
AATCAACAATGATGAAAAGCGATGCTCAGTCCTCTCACCGACCCGCCCACCTTCCCT
CTGTTTCATCTTATACATATTTCCCAGGCATCTGTTCTGCTATTACGTGCTATTATGTCTGAT
AATAGTATGAAAAGAAACAAAACTTTAAAGGACACAAAAGGAAGGACCACA
GGGAATGCCTGATGCTGTTAGAGTCTTTAGGGCCATGAAAGGAAAAATGAAGTCTA
GTGTATATAAAAATTCCTTAATTCTGCAGTTCTTTTAAAAAAAAAGCGTAAAAATTA
TGTGGTTGGTTTGTTTGTTTGTTTGTTTGTTTTTCTAATAATGATTTTTAAGGTAGGAAGGAT
TTCAGGCTCTATTTACATAATTGTTCTTTCCTTTTCACACAGAATCCCTTTTTA
GAAGTCAAGGTGACAGACACACCCAAGAGGTCCCGGAGAGACTTTGGGCTTGACTG
CGATGAGCACTCCACGGAATCCCGGTGCTGCCGCTACCCCCTCACGGTCGATTTTGA
AGCCTTTGGATGGGACTGGATTATCGCACCCAAAAGATATAAGGCCAATTACTGCTC
AGGAGAGTGTGAATTTGTGTTTTTACAAAAATATCCGCATACTCATCTTGTGCACCA
AGCAAACCCCAGAGGCTCAGCAGGCCCTTGCTGCACTCCGACAAAAATGTCTCCCA
TTAATATGCTATATTTTAATGGCAAAGAACAAATAATATATGGGAAAATTCCAGCCA
TGGTAGTAGACCGCTGTGGGTGCTCAGAAGCTGCGGCAAAAGAAGCAGCGGCTAAA
GAAGCGGCGGCAAAAGGAGTGCAGGTGGAAACCATCTCCCCAGGAGACGGGCGCA
CCTTCCCCAAGCGCGGCCAGACCTGCGTGGTGCACTACACCGGGATGCTTGAAGAT
GGAAAGAAAGTTGATTCCTCCCGGGACAGAAACAAGCCCTTTAAGTTTATGCTAGG
CAAGCAGGAGGTGATCCGAGGCTGGGAAGAAGGGGTTGCCCAGATGAGTGTGGGTC
AGAGAGCCAAACTGACTATATCTCCAGATTATGCCTATGGTGCCACTGGGCACCCAG
GCATCATCCCACCACATGCCACTCTCGTCTTCGATGTGGAGCTTCTAAAACTGGAAG
AAGCTGCGGCAAAAGAAGCAGCGGCTAAAGAAGCGGCGGCAAAAGTGAGCAAGGG
CGAGGAGGATAACATGGCCATCATCAAGGAGTTCATGCGCTTCAAGGTGCACATGG
AGGGCTCCGTGAACGGCCACGAGTTCGAGATCGAGGGCGAGGGCGAGGGCCGCCCC
TACGAGGGCACCCAGACCGCCAAGCTGAAGGTGACCAAGGGTGGCCCCCTGCCCTT
CGCCTGGGACATCCTGTCCCCTCAGTTCATGTACGGCTCCAAGGCCTACGTGAAGCA
CCCCGCCGACATCCCCGACTACTTGAAGCTGTCCTTCCCCGAGGGCTTCAAGTGGGA
GCGCGTGATGAACTTCGAGGACGGCGGCGTGGTGACCGTGACCCAGGACTCCTCCC
TGCAGGACGGCGAGTTCATCTACAAGGTGAAGCTGCGCGGCACCAACTTCCCCTCC
GACGGCCCCGTAATGCAGAAGAAAACCATGGGCTGGGAGGCCTCCTCCGAGCGGAT
GTACCCCGAGGACGGCGCCCTGAAGGGCGAGATCAAGCAGAGGCTGAAGCTGAAG
GACGGCGGCCACTACGACGCTGAGGTCAAGACCACCTACAAGGCCAAGAAGCCCGT
GCAGCTGCCCGGCGCCTACAACGTCAACATCAAGTTGGACATCACCTCCCACAACG
AGGACTACACCATCGTGGAACAGTACGAACGCGCCGAGGGCCGCCACTCCACCGGC
GGCATGGACGAGCTGTACAAGTAAGCTTTGCATTAGGTTAGAAATTTCGCAAGTCAT
GGAAGGTCTTCCCCTCAATTTCGAAACTGTGAATTCAAGCACCACAGGCTGTAGGCC
TTGAGTATGCTCTAGTAACGTAAGCACAAGCTACAGTGTATGAACTAAAAGAGAGA
ATAGATGCAATGGTTGGCATTCAACCACCAAAATAAACCATACTATAGGATGTTGTA
TGATTTCCAGAGTTTTTGAAATAGATGGAGATCAAATTACATTTATGTCCATATATG
TATATTACAACTACAATCTAGGCAAGGAAGTGAGAGCACATCTTGTGGTCTGCTGAG
TTAGGAGGGTATGATTAAAAGGTAAAGTCTTATTTCCTAACAGTTTCACTTAATATT
TACGGAAGAATCTATATGTAGCCTTTGTAAAGTGTAGGATTGTTATCATTTAAAAAC
ATCATGTACACTTATATTTGTATTGTATACTTGGTAAGATAAAATTCCACAAAGTAG
GAATGGGGCCTTACATACACATTGCCATTCCTATTATAATTGGACAATCCACCACGG
TGCTAATGCAGTGCTGAATGGCTCCTACTGGACCTCTCGATAGAACACTCTACAAAG
TACGAGTCTCTCTCTCCCTTCCAGGTGCATCTCCACACACACAGCACTAAGTGTTCA
ATGCATTTTCTTTAAGGAAAGAAGAATCTTTTTTTCTAGAGGTCAACTTTCAGTCAAC
TCTAGCACAGCGGGAGTGACTGCTGCATCTTAAAAGGCAGCCAAACAGTATTCATTT
TTTAATCTAAATTTCAAAATCACTGTCTGCCTTTATCACATGGCAATTTTGTGGTAAA
ATAATGGAAATGACTGGTTCTATCAATATTGTATAAAAGACTCTGAAACAATTACAT
TTATATAATATGTATACAATATTGTTTTGTAAATAAGTGTCTCCTTTTATATTTACTTT
GGTATATTTTTACACTAATGAAATTTCAAATCATTAAAGTACAAAGACATGTCATGT
ATCACAAAAAAGGTGACTGCTTCTATTTCAGAGTGAATTAGCAGATTCAATAGTGGT
CTTAAAACTCTGTATGTTAAGATTAGAAGGTTATATTACAATCAATTTATGTATTTTT
TACATTATCAACATTCACTTATGGTTTCATGGTGGCTGTATCTATGAATGTGGCTCCC
AGTCAAATTTCAATGCCCCACCATTTTAAAAATTACAAGCATTACTAAACATACCAA
CATGTATCTAAAGAAATACAAATATGGTATCTCAATAACAGCTACTTTTTTATTTTAT
AATTTGACAATGAATACATTTCTTTTATTTACTTCAGTTTTATAAATTGGAACTTTGT
TTATCAAATGTATTGTACTCATAGCTAAATGAAATTATTTCTTACATAAAAATGTGT
AGAAACTATAAATTAAAGTGTTTTCACATTTTTGAAAGGCATCAGTTTTATGTCATA
ATGATTAATTGTGGGTTTTTAAATTTTTATTTTATTATAAGTTTGTACAAAAGTTTGT
ACCAAAAAGGTTAGTACAGAAGTTTGATTTAGGAACTCTAGTTCTGAGTGAAATTCC
CAATGAATTTGCTTTATATATACAAAAGCAAAGACTAGTGTCTCCTGCTCCATTTTTC
TCTAACTTGAGCTAAAATCTATTTAATTTCATAGGGTCGATTTTAAATGCATTTCAAT
TGGCAGTGGTATATACTCCTAGGGCTACATACTCCACCGAAGTGACAGGGACACAG
GAAACCACAGTTCTTCTCTAACACTTAATGAGACTGGCACACTTTCCTGAGCACTGG
GGCCACCAGTTTTCAAACAGAATAGCAGAGTGACTTAAATTACCAAGAAGTCCTTGT
GTACCATTGAGCAGGAGTATATCTAAGCCCTTCTGTACCACTGTGTAGGAGTATGTC
TGAGCAATTTCTTCCTTCTACCTTCATCAGGTCAGGGATGTGGCCCTACAGTGACTTA
AGGTTAAAGGGGAGGTGGCGGCCGCATTCTGGTACCAGGGCCCATCAAGCTTGCTA
CGTACGTCGACAGCTTGATATCGAATTCCGCTAGCTTGGCTGCAGGTCGTCGAAATC
TGTTGACAATTAATCATCGGCATAGTATATCGGCATAGTATAATACGACAAGGTGAG
GAACTAAACCATGGGATCGGCCATTGAACAAGATGGATTGCACGCAGGTTCTCCGG
CCGCTTGGGTGGAGAGGCTATTCGGCTATGACTGGGCACAACAGACAATCGGCTGC
TCTGATGCCGCCGTGTTCCGGCTGTCAGCGCAGGGGCGCCCGGTTCTTTTTGTCAAG
ACCGACCTGTCCGGTGCCCTGAATGAACTGCAGGACGAGGCAGCGCGGCTATCGTG
GCTGGCCACGACGGGCGTTCCTTGCGCAGCTGTGCTCGACGTTGTCACTGAAGCGGG
AAGGGACTGGCTGCTATTGGGCGAAGTGCCGGGGCAGGATCTCCTGTCATCTCACCT
TGCTCCTGCCGAGAAAGTATCCATCATGGCTGATGCAATGCGGCGGCTGCATACGCT
TGATCCGGCTACCTGCCCATTCGACCACCAAGCGAAACATCGCATCGAGCGAGCAC
GTACTCGGATGGAAGCCGGTCTTGTCGATCAGGATGATCTGGACGAAGAGCATCAG
GGGCTCGCGCCAGCCGAACTGTTCGCCAGGCTCAAGGCGCGCATGCCCGACGGCGA
TGATCTCGTCGTGACCCATGGCGATGCCTGCTTGCCGAATATCATGGTGGAAAATGG
CCGCTTTTCTGGATTCATCGACTGTGGCCGGCTGGGTGTGGCGGACCGCTATCAGGA
CATAGCGTTGGCTACCCGTGATATTGCTGAAGAGCTTGGCGGCGAATGGGCTGACC
GCTTCCTCGTGCTTTACGGTATCGCCGCTCCCGATTCGCAGCGCATCGCCTTCTATCG
CCTTCTTGACGAGTTCTTCTGAGGGGATCAATTCTCTAGAGCTCGCTGATCAGCCTC
GACTGTGCCTTCTAGTTGCCAGCCATCTGTTGTTTGCCCCTCCCCCGTGCCTTCCTTG
ACCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACAGCAA
GGGGGAGGATTGGGAAGACAATAGCAGGCATGCTGGGGATGCGGTGGGCTCTATGG
CTTCTGAGGCGGAAAGAACCAGCTGGGGCTCGACTAGAGCTTGCGGAACCCTTCAT
CCACCCCTAGGAGGTATCGCGACGGATGGATCCAAGAACCAGCCCGGGCGGTGGAG
CTCCAGCTTTTGTTCCCTTTAGTGAGGGTTAATTTCGAGCTTGGCGTAATCATGGTCA
TAGCTGTTTCCTGTGTGAAATTGTTATCCGCTCACAATTCCACACAACATACGAGCC
GGAAGCATAAAGTGTAAAGCCTGGGGTGCCTAATGAGTGAGCTAACTCACATTAAT
TGCGTTGCGCTCACTGCCCGCTTTCCAGTCGGGAAACCTGTCGTGCCAGCTGCATTA
ATGAATCGGCCAACGCGCGGGGAGAGGCGGTTTGCGTATTGGGCGCTCTTCCGCTTC
CTCGCTCACTGACTCGCTGCGCTCGGTCGTTCGGCTGCGGCGAGCGGTATCAGCTCA
CTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGC
GTTTTTCCATAGGCTCCGCCCCCCTGACGAGCATCACAAAAATCGACGCTCAAGTCA
GAGGTGGCGAAACCCGACAGGACTATAAAGATACCAGGCGTTTCCCCCTGGAAGCT
CCCTCGTGCGCTCTCCTGTTCCGACCCTGCCGCTTACCGGATACCTGTCCGCCTTTCT
CCCTTCGGGAAGCGTGGCGCTTTCTCATAGCTCACGCTGTAGGTATCTCAGTTCGGT
GTAGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTATC
GCCACTGGCAGCAGCCACTGGTAACAGGATTAGCAGAGCGAGGTATGTAGGCGGTG
CTACAGAGTTCTTGAAGTGGTGGCCTAACTACGGCTACACTAGAAGAACAGTATTTG
GTATCTGCGCTCTGCTGAAGCCAGTTACCTTCGGAAAAAGAGTTGGTAGCTCTTGAT
CCGGCAAACAAACCACCGCTGGTAGCGGTGGTTTTTTTGTTTGCAAGCAGCAGATTA
CGCGCAGAAAAAAAGGATCTCAAGAAGATCCTTTGATCTTTTCTACGGGGTCTGACG
CTCAGTGGAACGAAAACTCACGTTAAGGGATTTTGGTCATGAGATTATCAAAAAGG
ATCTTCACCTAGATCCTTTTAAATTAAAAATGAAGTTTTAAATCAATCTAAAGTATA
TATGAGTAAACTTGGTCTGACAGTTACCAATGCTTAATCAGTGAGGCACCTATCTCA
GCGATCTGTCTATTTCGTTCATCCATAGTTGCCTGACTCCCCGTCGTGTAGATAACTA
CGATACGGGAGGGCTTACCATCTGGCCCCAGTGCTGCAATGATACCGCGAGACCCA
CGCTCACCGGCTCCAGATTTATCAGCAATAAACCAGCCAGCCGGAAGGGCCGAGCG
CAGAAGTGGTCCTGCAACTTTATCCGCCTCCATCCAGTCTATTAATTGTTGCCGGGA
AGCTAGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTTCCCA
ACGATCAAGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTGCTCT
TGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTC
ACCAGCGTTTCTGGGTGAGCAAAAACAGGAAGGCAAAATGCCGCAAAAAAGGGAA
TAAGGGCGACACGGAAATGTTGAATACTCATACTCTTCCTTTTTCAATATTATTGAA
GCATTTATCAGGGTTATTGTCTCATGAGCGGATACATATTTGAATGTATTTAGAAAAATAAAC
AAATAGGGGTTCCGCGCACATTTCCCCGAAAAGTGCCAC

4. Preparation of Injection Mixture

Tube 1 solution: 0.8 μL of 100 picomole per microliter (pmol/μL) gRNA is added to RNase-free water, followed by the addition of 0.2 μL of 20 μM Cas9 protein (NEB, catalog number: MO646M), the mixture is mixed well, and then incubated at 25° C. in a metal bath for 10 min to obtain Tube 1 solution;

Tube 2 solution is Donor recombinant vector with a final concentration of 15 nanogram per microliter (ng/μL);

Tube 1 solution and Tube 2 solution are mixed to obtain the injection mixture, with a total volume of 20 μL.

5. Microinjection and embryo transplantation

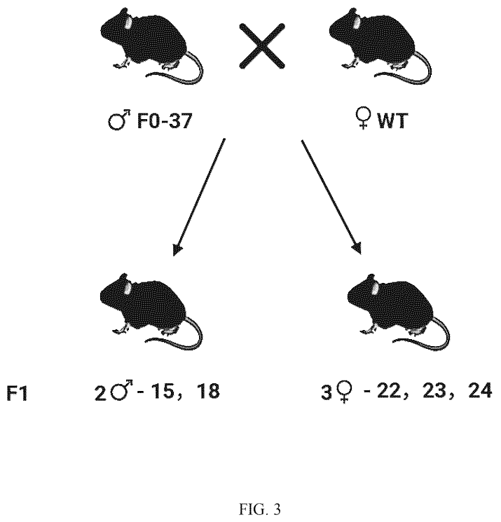

After superovulation of female C57BL/6J mice, the fertilized eggs of C57BL/6J mice are obtained by in vitro fertilization; then, the obtained injection mixture is injected into the fertilized eggs of C57BL/6J mice by using a glass microinjection needle with an extremely fine tip (0.1-0.5 micrometers (μM)), and then the fertilized eggs after micro-injection are transferred to the fallopian tubes of surrogate C57BL/6J mice through embryo transplantation, and the F0 generation mice are obtained after the young mice are born. The genotype of F0 generation mice is identified by PCR, as shown in FIG. 3.

The specific operation for embryo transplantation is as follows: the fertilized eggs after microinjection are cultured in vitro to the morula stage, followed by quality testing; a small opening is cut in the capsule of the fallopian tube of the surrogate mother mouse; after clearly observing the entire path of the oviduct, the transplantation tube 2-3 millimeters (mm) is inserted into the opening and the well-developed embryos are blown into the ampulla, then pause briefly before withdrawing the transplantation tube.

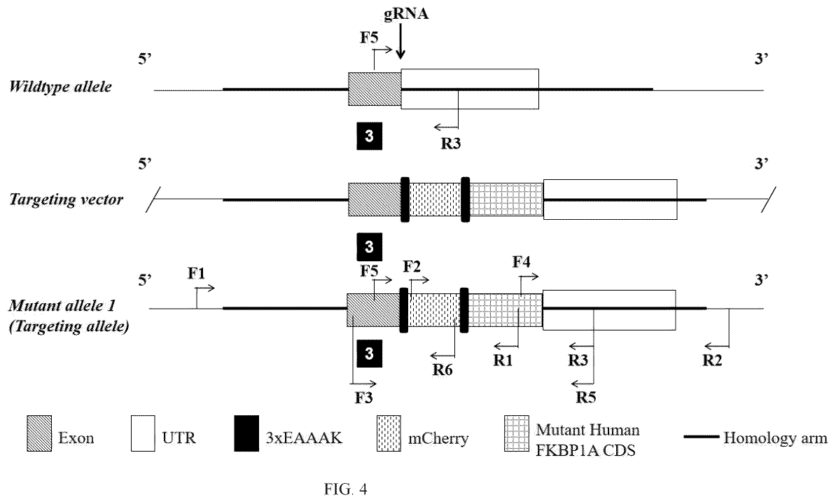

6. The positive F0 generation mice are mated with C57BL/6J mice (Wildtype, Wt) to obtain F1 generation mice, and genotyping PCR identification is performed on them. If positive mice (F1 generation heterozygotes) are born, it indicates that the knock-in sequence has been integrated into the germ cells, as shown in FIG. 4.

7. The F1-generation heterozygous mice are intercrossed, and genotyping PCR identification are performed on the resulting F2-generation mice to obtain MSTN-FKBP-mCherry homozygous knock-in mice, that is, mice expressing the MSTN-FKBP-mCherry fusion protein.

8. By administration of dTAG to the obtained knock-in mice in the later stage enables rapid and reversible degradation and clearance of the MSTN protein in the mice.

### Embodiment 2

### Results of Mouse Genotyping PCR Identification

### 1. DNA Extraction

A newborn mouse aged 3-4 weeks is selected, approximately 0.3 centimeters (cm) of the mouse tail is cut, and place it in a 1.5 milliliters (mL) EP tube (marked with the corresponding mouse number on the tube cap) containing 50 μL lysis buffer and 1 μL protease k, the tail is centrifuged at the bottom of the EP tube, and incubated in a 55° C. metal bath overnight. The next day, the metal bath is set to 95° C. for 5 min to inactivate the protease k, and the vortex shaker is oscillated for 15 seconds (s) to ensure complete lysis of the tail, then the supernatant is centrifuged at 12,000 revolutions per minute (rpm) for 5 min. The supernatant may be directly used as a template for genotyping or stored at −20° C. for future use.

### 2. Genotyping PCR Identification Results of F1 Generation Mice

The DNA of mouse tail tip is extracted and genotyping PCR amplification is performed. According to the PCR identification strategy shown in FIG. 4, using primers F1/R1 for amplification, the 5′HA homologous recombination-positive genome should amplify a 2.8 kilobase (kb) fragment, while the negative genome should show no band; using primers F2/R2 for amplification, the 3′HA homologous recombination-positive genome should amplify a 3.5 kb fragment, while the negative genome should show no band. The amplification system is shown in Table 9, and the amplification program is shown in Table 10. The specific primers for PCR identification are shown in Table 11.

TABLE 9
PCR system

| Component | Amount (μL) |
|---|---|
| Mouse DNA | 1 |
| Forward primer F (10 μM) | 1 |
| Reverse primer F (10 μM) | 1 |
| Premix Taq Polymerase | 12.5 |
| ddH₂O | 9.5 |
| Total | 25 |

TABLE 10
PCR program

| Step | Temperature (° C.) | Time | Cycles |
|---|---|---|---|
| Pre-denaturation | 94 | 3 min | 1 |
| Denaturation | 94 | 30 s | 35 |
| Annealing | 60 | 35 s | |
| Extension | 72 | 35 s | |
| Final extension | 72 | 5 min | 1 |
| Storage | 4 | ∞ | 1 |

TABLE 11
PCR amplification primers

| Primer | | | |
|---|---|---|---|
| Name | Sequence (5′-3′) | Primer Type | Sequence Number |
| F1 | CCTTCCCGTTTTATGTTTCTTTCCT | Forward primer | SEQ ID NO. 11 |
| R1 | ATGGTTTTCTTCTGCATTACGGG | Reverse primer | SEQ ID NO. 12 |
| F2 | GAGTGCAGGTGGAAACCATCTC | Forward primer | SEQ ID NO. 13 |
| R2 | TTTTCCCCAAGACTTTACAAACCC | Reverse primer | SEQ ID NO. 14 |

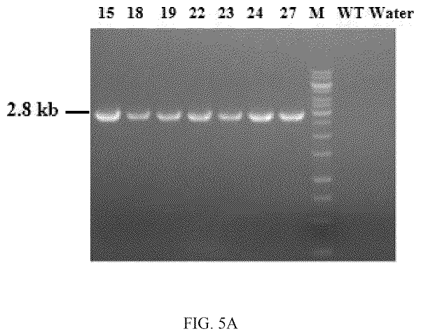

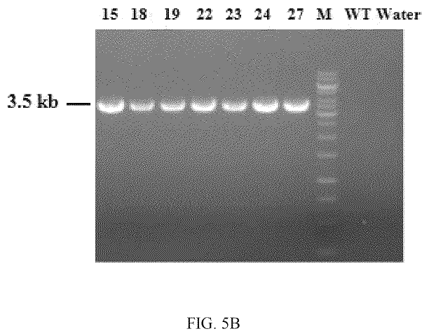

The identification results are shown in FIG. 5A and FIG. 5B. Mice numbered 15, 18, 19, 22, 23, 24, and 27 are F1 generation mice positive for bilateral homologous recombination.

Note: if the DNA sample is not very pure or lacks sufficient PCR extension time, long-fragment PCR products may not be amplified. The alternative PCR identification strategy in FIG. 4 may be used, with primers F4/R5 for amplification. The target allele should amplify a 460 bp fragment. The sequences of primers F4/R5 are shown in Table 13.

### 3. Sequencing Results of the Knock-In Sequence in F1 Generation Mice

The PCR amplification is performed using primers F3/R3, and the resulting product is submitted for sequencing.

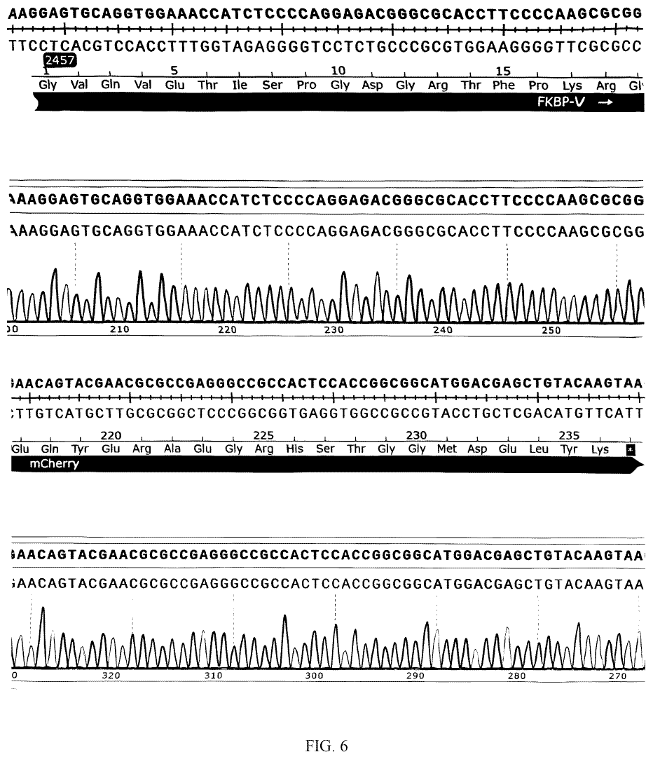

The sequencing results are shown in FIG. 6. The results indicate that the knock-in site and knock-in sequence in the positive F1 generation No. 15 mouse are correct, with no mutations. The specific primers for PCR amplification are shown in Table 12:

TABLE 12
PCR amplification primers

| Primer | | | |
|---|---|---|---|
| Name | Sequence (5′-3′) | Primer Type | Sequence Number |
| F3 | GACTGGATTATCGCACCCAAAAG | Forward primer | SEQ ID NO. 15 |
| R3 | GCTCTCACTTCCTTGCCTAGATTG | Reverse primer | SEQ ID NO. 16 |

### 4. Genotyping PCR Identification Results of F2 Generation Mice

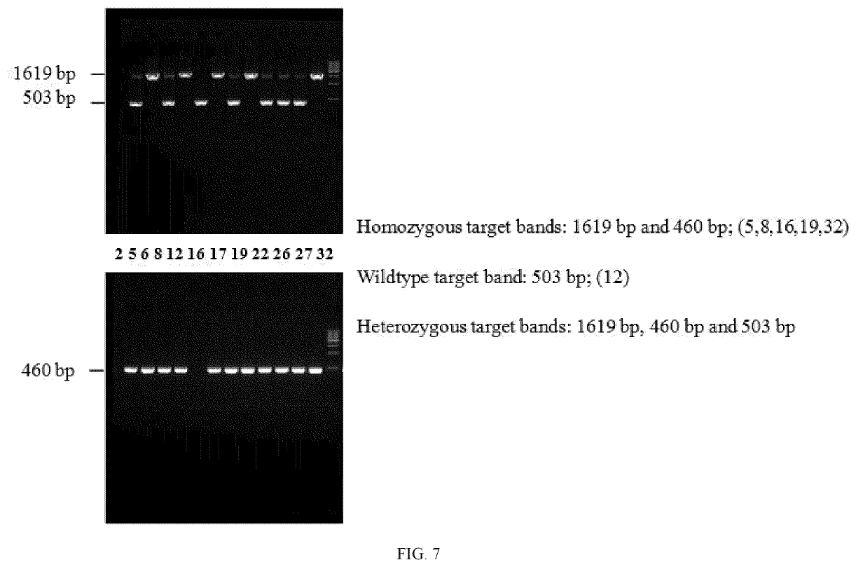

The DNA from mouse tail tips is extracted and performed genotyping PCR amplification. According to the PCR identification strategy in FIG. 4, primers F5/R3 and F4/R5 are used for PCR. The genotypes of F2 generation mice are divided into three categories based on PCR fragment lengths: knock-in homozygotes (Homozygous, Ho), knock-in heterozygotes (Heterozygous, He), and wild-type (Wildtype, Wt). The PCR fragment lengths corresponding to different genotypes are as follows: 460 bp and 1619 bp bands (knock-in homozygotes); 460 bp, 503 bp, and 1619 bp bands (knock-in heterozygotes); and 503 bp band (wild-type); as shown in FIG. 7. The specific primers for PCR identification are shown in Table 13.

TABLE 13
PCR amplification primers

| Primer | | | |
|---|---|---|---|
| Name | Sequence (5′-3′) | Primer Type | Sequence Number |
| F5 | ACTGCTCAGGAGAGTGTGAATTT | Forward primer | SEQ ID NO. 19 |
| R3 | GCTCTCACTTCCTTGCCTAGATTG | Reverse primer | SEQ ID NO. 16 |
| F4 | TCAAGACCACCTACAAGGCCAA | Forward primer | SEQ ID NO. 17 |
| R5 | TCACTTCCTTGCCTAGATTGTAGTT | Reverse primer | SEQ ID NO. 18 |

### Embodiment 3

### Evaluation of the Targeting Strategy for Transient Knockout of MSTN

The expression of MSTN protein in muscle tissues of F2 generation mice is detected by Western blot.

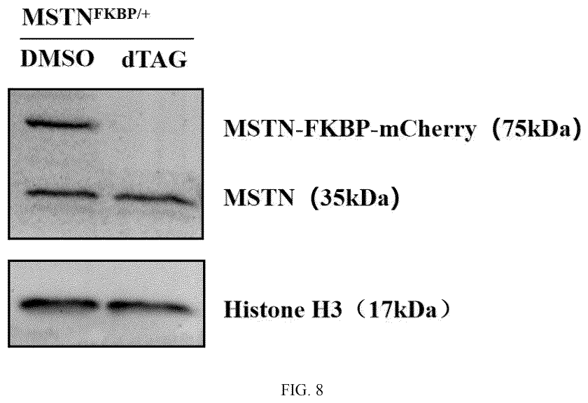

The identification results are shown in FIG. 8. After administering dTAG to F2 generation heterozygous mice, the MSTN-FKBP-mCherry fusion protein is degraded, while MSTN protein is normally expressed in muscle tissues of mice not administered with dTAG.

The above results indicate that the disclosure uses In-Fusion technology to construct the Donor vector; by co-injecting the Donor vector, gRNA, and Cas9 mRNA into mouse fertilized eggs, the human FKBP1A gene sequence is knocked into the mouse genome using CRISPR/Cas9-mediated gene editing technology, serving as the FKBP-mCherry tag sequence fused with the mouse Mstn gene; and through embryo transplantation and breeding of the obtained positive F0 generation mice, parents mice that stably and efficiently express the MSTN-FKBP-mCherry fusion protein are ultimately obtained. This enables the use of the dTAG protein rapid degradation system model mice to achieve rapid, complete, and reversible degradation of MSTN protein induced by small molecule degraders, which provides new ideas and approaches to address the challenge of traditional gene knockout animal models being unable to explore the direct regulatory mechanisms of proteins of interest.

1. A method for constructing a mouse model for rapid degradation of myostatin (MSTN) protein, comprising following steps:
inserting sequentially a 5′ homologous arm, 3x (Glu-Ala-Ala-Ala-Lys) (3xEAAAK), an FK506-binding protein (FKBP1A) gene synonymous mutation sequence, the 3xEAAAK, a red fluorescent protein monomeric Cherry (mCherry) sequence and a 3′ homologous arm into a Donor vector backbone to obtain a Donor recombinant vector; wherein a nucleotide sequence of the FKBP1A gene synonymous mutation sequence is as shown in Sequence Identification Number (SEQ ID NO.) 4, a nucleotide sequence of the 3xEAAAK is as shown in SEQ ID NO. 3, a nucleotide sequence of the red fluorescent protein mCherry sequence is as shown in SEQ ID NO. 5, a nucleotide sequence of the 5′ homologous arm is as shown in SEQ ID NO. 2, and a nucleotide sequence of the 3′ homologous arm is as shown in SEQ ID NO. 6; wherein a nucleotide sequence of the Donor recombinant vector is as shown in SEQ ID NO. 20;
designing a Guide Ribonucleic Acid (gRNA) based on a third exon of an Mstn gene on mouse chromosome 1, wherein a nucleotide sequence of the gRNA is as shown in SEQ ID NO. 1;
mixing the gRNA, CRISPR-associated protein 9 (Cas9) protein, and the Donor recombinant vector to yield a mixture and co-injecting the mixture into mouse fertilized eggs to knock the FKBP1A gene synonymous mutation sequence and the red fluorescent protein mCherry sequence into a mouse genome;
culturing the mouse fertilized eggs after injection in vitro and transplanting into surrogate mice to continue development until birth of young mice, thus obtaining F0 generation mice; and
hybridizing the F0 generation mice with wild-type mice, or further breeding hybridized offspring to obtain mice expressing Myostatin-FK506-binding protein-monomeric Cherry (MSTN-FKBP-mCherry) fusion protein, namely, the mouse model for the rapid degradation of the MSTN protein.

2. The method for constructing the mouse model for the rapid degradation of the MSTN protein according to claim 1, wherein the Donor recombinant vector is constructed by following steps:
using a Bacterial Artificial Chromosome (BAC) plasmid as a template, amplifying fragments comprising the 5′ homologous arm and the 3′ homologous arm from the mouse genome respectively to obtain a fragment-1 and a fragment-3;
connecting the 3xEAAAK, the FKBP1A gene synonymous mutation sequence, the 3xEAAAK, and the red fluorescent protein mCherry sequence sequentially to obtain a fragment-2; and
ligating the fragment-1, the fragment-2, the fragment-3, and the Donor vector backbone, incubating a resulting ligation product, transforming into competent cells, culturing, and extracting to obtain the Donor recombinant vector.

3. The method for constructing the mouse model for the rapid degradation of the MSTN protein according to claim 2, wherein primer sequences for amplifying the fragments comprising the 5′ homologous arm are as shown in SEQ ID NO. 7 and SEQ ID NO. 8, and primer sequences for amplifying the fragments comprising the 3′ homologous arm are as shown in SEQ ID NO. 9 and SEQ ID NO. 10.

4. The method for constructing the mouse model for the rapid degradation of the MSTN protein according to claim 2, wherein when the fragment-1, the fragment-2, the fragment-3, and the Donor vector backbone are connected, a 20 microliters (μL) ligation system comprises: 80.0 nanograms (ng) of the Donor vector backbone, 7.19 ng of the fragment-1, 21.28 ng of the fragment-2, 27.54 ng of the fragment-3, and 10 μL of NEBuilder® HiFi DNA Assembly MasterMix.

5. The method for constructing the mouse model for the rapid degradation of the MSTN protein according to claim 2, wherein the competent cells are DH5-alpha competent cells.

6. The method for constructing the mouse model for the rapid degradation of the MSTN protein according to claim 1, wherein the gRNA and the Cas9 protein are mixed and incubated in a metal bath, then the Donor recombinant vector is added, mixed evenly, and co-injected into the mouse fertilized eggs.

7. The method for constructing the mouse model for the rapid degradation of the MSTN protein according to claim 6, wherein a concentration of the gRNA is 90-110 picomoles per microliter (pmol/μL), a concentration of the Cas9 protein is 18-22 micromolar (μM), a concentration of the Donor recombinant vector is 12-18 nanograms per microliter (ng/μL), a volume ratio of the gRNA to the Cas9 protein is 3-5:1, and a total volume of the mixture of the gRNA and the Cas9 protein with the Donor recombinant vector is 20 μL.

8. A mouse model for the rapid degradation of the MSTN protein constructed by the method according to claim 1.

9. The mouse model for the rapid degradation of the MSTN protein according to claim 8, wherein obtained MSTN-FKBP-mCherry knock-in mice are administered with degradation tag (dTAG) to achieve reversible degradation and clearance of the MSTN protein in the mice.


SEQUENCE LISTING (20 sequences)

SEQ ID NO: 1 - DNA, 23 bp, synthetic construct
  source 1..23: mol_type = other DNA
gggaagaccttccatgacttggg

SEQ ID NO: 2 - DNA, 378 bp, synthetic construct
  source 1..378: mol_type = other DNA
aatccctttttagaagtcaaggtgacagacacacccaagaggtcccggagagactttgggcttgactgcgatgagcactccacggaatcccggtgctgccgctaccccctcacggtcgattttgaagcctttggatgggactggattatcgcacccaaaagatataaggccaattactgctcaggagagtgtgaatttgtgtttttacaaaaatatccgcatactcatcttgtgcaccaagcaaaccccagaggctcagcaggcccttgctgcactccgacaaaaatgtctcccattaatatgctatattttaatggcaaagaacaaataatatatgggaaaattccagccatggtagtagaccgctgtgggtgctca

SEQ ID NO: 3 - DNA, 45 bp, synthetic construct
  source 1..45: mol_type = other DNA
gaagctgcggcaaaagaagcagcggctaaagaagcggcggcaaaa

SEQ ID NO: 4 - DNA, 321 bp, synthetic construct
  source 1..321: mol_type = other DNA
ggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 5 - DNA, 708 bp, synthetic construct
  source 1..708: mol_type = other DNA
gtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 6 - DNA, 1448 bp, synthetic construct
  source 1..1448: mol_type = other DNA
gctttgcattaggttagaaatttcgcaagtcatggaaggtcttcccctcaatttcgaaactgtgaattcaagcaccacaggctgtaggccttgagtatgctctagtaacgtaagcacaagctacagtgtatgaactaaaagagagaatagatgcaatggttggcattcaaccaccaaaataaaccatactataggatgttgtatgatttccagagtttttgaaatagatggagatcaaattacatttatgtccatatatgtatattacaactacaatctaggcaaggaagtgagagcacatcttgtggtctgctgagttaggagggtatgattaaaaggtaaagtcttatttcctaacagtttcacttaatatttacggaagaatctatatgtagcctttgtaaagtgtaggattgttatcatttaaaaacatcatgtacacttatatttgtattgtatacttggtaagataaaattccacaaagtaggaatggggccttacatacacattgccattcctattataattggacaatccaccacggtgctaatgcagtgctgaatggctcctactggacctctcgatagaacactctacaaagtacgagtctctctctcccttccaggtgcatctccacacacacagcactaagtgttcaatgcattttctttaaggaaagaagaatctttttttctagaggtcaactttcagtcaactctagcacagcgggagtgactgctgcatcttaaaaggcagccaaacagtattcattttttaatctaaatttcaaaatcactgtctgcctttatcacatggcaattttgtggtaaaataatggaaatgactggttctatcaatattgtataaaagactctgaaacaattacatttatataatatgtatacaatattgttttgtaaataagtgtctccttttatatttactttggtatatttttacactaatgaaatttcaaatcattaaagtacaaagacatgtcatgtatcacaaaaaaggtgactgcttctatttcagagtgaattagcagattcaatagtggtcttaaaactctgtatgttaagattagaaggttatattacaatcaatttatgtattttttacattatcaacattcacttatggtttcatggtggctgtatctatgaatgtggctcccagtcaaatttcaatgccccaccattttaaaaattacaagcattactaaacataccaacatgtatctaaagaaatacaaatatggtatctcaataacagctacttttttattttataatttgacaatgaatacatttcttttatttacttcagttttataaattggaactttgtttatcaaatgtattgtactcatagctaaatgaaattatttcttacataaaaatgtgtagaaactataaattaaagtgttttcacatttttgaaaggc

SEQ ID NO: 7 - DNA, 31 bp, synthetic construct
  source 1..31: mol_type = other DNA
ttccttttcacacagaatccctttttagaag

SEQ ID NO: 8 - DNA, 30 bp, synthetic construct
  source 1..30: mol_type = other DNA
ttcttttgccgcagcttctgagcacccaca

SEQ ID NO: 9 - DNA, 31 bp, synthetic construct
  source 1..31: mol_type = other DNA
gagctgtacaagtaagctttgcattaggtta

SEQ ID NO: 10 - DNA, 37 bp, synthetic construct
  source 1..37: mol_type = other DNA
attgaacacttagtgctgtgtgtgtggagatgcacct

SEQ ID NO: 11 - DNA, 25 bp, synthetic construct
  source 1..25: mol_type = other DNA
ccttcccgttttatgtttctttcct

SEQ ID NO: 12 - DNA, 23 bp, synthetic construct
  source 1..23: mol_type = other DNA
atggttttcttctgcattacggg

SEQ ID NO: 13 - DNA, 22 bp, synthetic construct
  source 1..22: mol_type = other DNA
gagtgcaggtggaaaccatctc

SEQ ID NO: 14 - DNA, 24 bp, synthetic construct
  source 1..24: mol_type = other DNA
ttttccccaagactttacaaaccc

SEQ ID NO: 15 - DNA, 23 bp, synthetic construct
  source 1..23: mol_type = other DNA
gactggattatcgcacccaaaag

SEQ ID NO: 16 - DNA, 24 bp, synthetic construct
  source 1..24: mol_type = other DNA
gctctcacttccttgcctagattg

SEQ ID NO: 17 - DNA, 22 bp, synthetic construct
  source 1..22: mol_type = other DNA
tcaagaccacctacaaggccaa

SEQ ID NO: 18 - DNA, 25 bp, synthetic construct
  source 1..25: mol_type = other DNA
tcacttccttgcctagattgtagtt

SEQ ID NO: 19 - DNA, 23 bp, synthetic construct
  source 1..23: mol_type = other DNA
actgctcaggagagtgtgaattt

SEQ ID NO: 20 - DNA, 9073 bp, synthetic construct
  source 1..9073: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacggcgcgccaagctaacctttgcctcctagcttatctttggactcctttagcatacttgcataggaattaacttaggcattaccaattaactaaatagtgagtcagtgataggacaagacctcatatattataaaaataataaaaaaatatttagccctccaaaacctgagttactgttctctgttacttttatttatttatttatcggctttcaaaaacttaacgtgaacatatggttatttccatccactgtcatagtttacaccaaaatattgtacttagaaataataagcctggttaccttttaaccttttgattgaacttgggaatatacagtctgggaatcagatctgttgtgagtgaaacctgtcatttgaagtactacgatttcctaaatgatctattttctttatatatgctacttaaacactacactctacaaattatgttctctttgacaatatagtctcatggtaaaagataaaattgctatcaactatttaagaacattctttctcatagacagtattttaattaccaaggttgctaacaggttttaattatttacaaggtggttagtgtgatattccttagagttttctctctaaaaggaaatggtcctaaatgcactgggagatggaagcggtcatgctgcatgcaaattcttctcttccttctgttccttctgctgtggtcctgaggattgacccagaaccttgcacatgacgggcaaaccctgttccaccaagctccattcactcccagttcatcacttcttataaacctcttctttgctcctttaaggattctctttccacgcactatacatacagccactgtcatcatgctaatttctctcttcccactgtaatccatatagtgctcagaaagagctactttgctctgactttaatatgcacatttctttcagaaaagacctaggggagaccagatgcgtacctatccatccaagagccattagtcacctgcaatcaacaatgatgaaaagcgatgctcagtcctctcaccgacccgcccaccttccctctgtttcatcttatacatatttcccaggcatctgttctgctattacgtgctattatgtctgataatagtatgaaaagaaacaaaactttaaaggacacaaaaggaaggaccacagggaatgcctgatgctgttagagtctttagggccatgaaaggaaaaatgaagtctagtgtatataaaaattccttaattctgcagttcttttaaaaaaaaagcgtaaaaattatgtggttggtttgtttgtttgtttgtttgtttttctaataatgatttttaaggtaggaaggatttcaggctctatttacataattgttctttccttttcacacagaatccctttttagaagtcaaggtgacagacacacccaagaggtcccggagagactttgggcttgactgcgatgagcactccacggaatcccggtgctgccgctaccccctcacggtcgattttgaagcctttggatgggactggattatcgcacccaaaagatataaggccaattactgctcaggagagtgtgaatttgtgtttttacaaaaatatccgcatactcatcttgtgcaccaagcaaaccccagaggctcagcaggcccttgctgcactccgacaaaaatgtctcccattaatatgctatattttaatggcaaagaacaaataatatatgggaaaattccagccatggtagtagaccgctgtgggtgctcagaagctgcggcaaaagaagcagcggctaaagaagcggcggcaaaaggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaagaagctgcggcaaaagaagcagcggctaaagaagcggcggcaaaagtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagctttgcattaggttagaaatttcgcaagtcatggaaggtcttcccctcaatttcgaaactgtgaattcaagcaccacaggctgtaggccttgagtatgctctagtaacgtaagcacaagctacagtgtatgaactaaaagagagaatagatgcaatggttggcattcaaccaccaaaataaaccatactataggatgttgtatgatttccagagtttttgaaatagatggagatcaaattacatttatgtccatatatgtatattacaactacaatctaggcaaggaagtgagagcacatcttgtggtctgctgagttaggagggtatgattaaaaggtaaagtcttatttcctaacagtttcacttaatatttacggaagaatctatatgtagcctttgtaaagtgtaggattgttatcatttaaaaacatcatgtacacttatatttgtattgtatacttggtaagataaaattccacaaagtaggaatggggccttacatacacattgccattcctattataattggacaatccaccacggtgctaatgcagtgctgaatggctcctactggacctctcgatagaacactctacaaagtacgagtctctctctcccttccaggtgcatctccacacacacagcactaagtgttcaatgcattttctttaaggaaagaagaatctttttttctagaggtcaactttcagtcaactctagcacagcgggagtgactgctgcatcttaaaaggcagccaaacagtattcattttttaatctaaatttcaaaatcactgtctgcctttatcacatggcaattttgtggtaaaataatggaaatgactggttctatcaatattgtataaaagactctgaaacaattacatttatataatatgtatacaatattgttttgtaaataagtgtctccttttatatttactttggtatatttttacactaatgaaatttcaaatcattaaagtacaaagacatgtcatgtatcacaaaaaaggtgactgcttctatttcagagtgaattagcagattcaatagtggtcttaaaactctgtatgttaagattagaaggttatattacaatcaatttatgtattttttacattatcaacattcacttatggtttcatggtggctgtatctatgaatgtggctcccagtcaaatttcaatgccccaccattttaaaaattacaagcattactaaacataccaacatgtatctaaagaaatacaaatatggtatctcaataacagctacttttttattttataatttgacaatgaatacatttcttttatttacttcagttttataaattggaactttgtttatcaaatgtattgtactcatagctaaatgaaattatttcttacataaaaatgtgtagaaactataaattaaagtgttttcacatttttgaaaggcatcagttttatgtcataatgattaattgtgggtttttaaatttttattttattataagtttgtacaaaagtttgtaccaaaaaggttagtacagaagtttgatttaggaactctagttctgagtgaaattcccaatgaatttgctttatatatacaaaagcaaagactagtgtctcctgctccatttttctctaacttgagctaaaatctatttaatttcatagggtcgattttaaatgcatttcaattggcagtggtatatactcctagggctacatactccaccgaagtgacagggacacaggaaaccacagttcttctctaacacttaatgagactggcacactttcctgagcactggggccaccagttttcaaacagaatagcagagtgacttaaattaccaagaagtccttgtgtaccattgagcaggagtatatctaagcccttctgtaccactgtgtaggagtatgtctgagcaatttcttccttctaccttcatcaggtcagggatgtggccctacagtgacttaaggttaaaggggaggtggcggccgcattctggtaccagggcccatcaagcttgctacgtacgtcgacagcttgatatcgaattccgctagcttggctgcaggtcgtcgaaatctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttcatccacccctaggaggtatcgcgacggatggatccaagaaccagcccgggcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac